(19) United States
(12) Patent Application Publication
(10) Pub. No.: US 2022/0072036 A1
(43) Pub. Date: Mar. 10, 2022

(54) METHOD FOR INHIBITING CORONAVIRUS AND METHOD FOR TREATING DISEASE ASSOCIATED WITH CORONAVIRUS INFECTION

(71) Applicants: Chung-Hsiang LIN, Taipei City (TW); AMAZING FORTUNE CO., LTD., Taipei City (TW)

(72) Inventors: Bo-Lin LIN, Taipei City (TW); Chung-Hsiang LIN, Taipei City (TW)

(21) Appl. No.: 17/468,293

(22) Filed: Sep. 7, 2021

Related U.S. Application Data
(60) Provisional application No. 63/075,557, filed on Sep. 8, 2020.

(51) Int. Cl. A61K 33/30; A61K 31/203; A61K 33/06; A61P 31/14

(52) U.S. Cl. CPC A61K 33/30; A61P 31/14; A61K 33/06; A61K 31/203

(57) ABSTRACT

Disclosed herein is a method for inhibiting a coronavirus, which includes administering to a subject in need thereof a retinoic acid and at least one bivalent metal ion. Also disclosed is a method for treating a disease associated with coronavirus infection, which includes administering to a subject in need thereof a retinoic acid and at least one bivalent metal ion.

### CROSS-REFERENCE TO RELATED APPLICATION

This application claims priority of U.S. Provisional Application No. 63/075,557, filed on Sep. 8, 2020.

### FIELD

The present disclosure relates to methods for inhibiting a coronavirus and for treating a disease associated with coronavirus infection, and more particularly to methods for inhibiting a coronavirus and for treating a disease associated with coronavirus infection using a combination of a retinoic acid and at least one bivalent metal ion.

### BACKGROUND

Coronaviruses are a group of positive-sense, single-strand RNA viruses belonging to the Coronaviridae family, which includes seven species/strains that infect humans, i.e., human coronavirus 0043 (HCoV-0043), human coronavirus HKU1 (HCoV-HKU1), human coronavirus 229E (HCoV-229E), human coronavirus NL63 (HCoV-NL63), Middle East respiratory syndrome-related coronavirus (MERS-CoV), severe acute respiratory syndrome coronavirus (SARS-CoV), and severe acute respiratory syndrome coronavirus 2 (SARS-CoV-2). Notably, SARS-CoV-2 is identified as the viral strain causing the current outbreak of coronavirus disease 2019 (COVID-19), the rapid spread of which was declared as a global pandemic known as COVID-19 pandemic. Symptoms of COVID-19 may be relatively non-specific, including fever, cough, fatigue, phlegm production, loss of sense of smell, shortness of breath, muscle and joint pain, headache, and chills, among others. Further development of COVID-19 symptoms may lead to complications, including breathing difficulties, pneumonia, acute respiratory distress syndrome, sepsis, septic shock, multi-organ failure, and death. Even though several COVID-19 vaccines have demonstrated high efficacy in preventing symptomatic COVID-19 infections during Phase III clinical trials, all potential adverse effects from such vaccines may not be known until use in general population (i.e., until post-marketing surveillance trials are conducted).

Drug repositioning (also known as drug repurposing) is the investigation of existing drugs for new therapeutic purposes. This research direction, along with development of COVID-19 vaccines and convalescent plasma transfusion, is being actively pursued to develop safe and effective COVID-19 treatments. In fact, several existing antiviral medications, previously developed or used in treatments for SARS, MERS, HIV/AIDS, and malaria, are being investigated as COVID-19 treatment candidates. A few of these medications, such as chloroquine and hydroxychloroquine, dexamethasone, favipiravir, lopinavir/ritonavir, remdesivir, etc., have advanced into clinical trials. However, based on published randomized controlled trials, none of these medications has yet been shown to be clearly effective in reducing mortality of COVID-19 patients. Therefore, there is still an urgent need to find other classes of drugs which are effective against SARS-CoV-2.

The SARS-CoV-2 genome shares high sequence identity with that of SARS-CoV. Both of SARS-CoV-2 and SARS-CoV critically rely on the activity of two viral proteases, namely, the main protease (Mpro, also known as 3CLpro or non-structural protein 5 (nsp5)) and the papain-like protease (PLpro, the protease domain of non-structural protein 3 (nsp3)), to achieve virus proliferation cycle and viral spread. PLpro is a potential target since such enzyme plays an essential role in cleavage and maturation of viral polyproteins, assembly of the replicase-transcriptase complex, and disruption of host responses. Even though the primary function of PLpro and 3CLpro is to process the viral polyprotein in a coordinated manner, PLpro has an additional function of stripping ubiquitin and IFN-stimulatory gene factor 15 (ISG15) from host-cell proteins to enable coronaviruses to avoid host innate immune responses (i.e. PLpro not only relates to viral replication, but also is associated with dysregulation of signaling cascades in infected cells which gives rise to cell death in surrounding, uninfected cells). Therefore, drugs are designed to target PLpro to fight against SARS-CoV-2.

### SUMMARY

Therefore, an object of the present disclosure is to provide a method for inhibiting a coronavirus, which can alleviate at least one of the drawbacks of the prior art, and which includes administering to a subject in need thereof a retinoic acid and at least one bivalent metal ion.

Another object of the present disclosure is to provide a method for treating a disease associated with coronavirus infection, which can alleviate at least one of the drawbacks of the prior art, and which includes administering to a subject in need thereof a retinoic acid and at least one bivalent metal ion.

### BRIEF DESCRIPTION OF THE DRAWINGS

Other features and advantages of the present disclosure will become apparent in the following detailed description of the embodiments with reference to the accompanying drawings, of which:

FIG. 1 shows the 50% tissue culture infectious dose (TCID₅₀) of each group in Example 2, infra, in which the symbol “*” represents p<0.05 compared with the control group, the symbol “**” represents p<0.01 compared with the control group, and the symbol “***” represents p<0.001 compared with the control group.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which this disclosure belongs. One skilled in the art will recognize many methods and materials similar or equivalent to those described herein, which could be used in the practice of this disclosure. Indeed, this disclosure is in no way limited to the methods and materials described.

In the development of anti-coronavirus drugs, the applicant surprisingly found that the combination of a retinoic acid and at least one bivalent metal ion can provide a synergistic effect on in vitro and in vivo inhibition of a coronavirus, and hence expected that such combination can serve as a potential drug-repurposing agent against a coronavirus, in particular, severe acute respiratory syndrome coronavirus 2 (SARS-CoV-2) that causes coronavirus disease 2019 (COVID-19).

Therefore, the present disclosure provides a method for inhibiting a coronavirus, which includes administering to a subject in need thereof a retinoic acid and at least one bivalent metal ion.

As used herein, the term “inhibiting a coronavirus” refers to reduction in the amount of coronavirus replication and complete arrest of coronavirus replication, or slowing, interrupting, arresting or stopping coronavirus infection.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

In certain embodiments, the retinoic acid is 13-cis-retinoic acid.

According to the present disclosure, the at least one bivalent metal ion may be selected from the group consisting of Zn²⁺, Mg²⁺, Cu²⁺, Mn²⁺, and combinations thereof. In certain embodiments, the at least one bivalent metal ion is Zn²⁺ or Mg²⁺. In an exemplary embodiment, the at least one bivalent metal ion is Zn²⁺. In another exemplary embodiment, the at least one bivalent metal ion is a combination of Zn²⁺ and Mg²⁺.

According to the present disclosure, the method may further include administering to the subject at least one monovalent metal ion selected from the group consisting of K⁺, Na⁺, and a combination thereof.

In certain embodiments, when the at least one bivalent metal ion is used in combination with the at least one monovalent metal ion, the at least one bivalent metal ion is a combination of Zn²⁺ and Mg²⁺, and the at least one monovalent metal ion is K⁺.

According to the present disclosure, the concentration of the at least one bivalent metal ion and the at least one monovalent metal ion may range from 1 mM to 600 mM, 1 mM to 400 mM, 1 mM to 200 mM, or from 10 mM to 150 mM.

According to the present disclosure, the coronavirus may be selected from the group consisting of severe acute respiratory syndrome coronavirus (SARS-CoV), severe acute respiratory syndrome coronavirus 2 (SARS-CoV-2), Middle East respiratory syndrome coronavirus (MERS-CoV), human coronavirus 229E (HcoV-229E), human coronavirus OC43 (HCoV-OC43), human coronavirus NL63 (HCoV-NL63), human coronavirus HKU (HCoV-HKU1), and combinations thereof. In an exemplary embodiment, the coronavirus is SARS-CoV-2.

According to the present disclosure, the retinoic acid and the at least one bivalent metal ion may be administered separately, simultaneously, or sequentially. In certain embodiments, the retinoic acid and the at least one bivalent metal ion are administered sequentially. In an exemplary embodiment, the at least one bivalent metal ion is administered before the retinoic acid is administered, and a time interval between administration of the at least one bivalent metal ion and that of the retinoic acid is at least one hour.

According to the present disclosure, the retinoic acid, the at least one bivalent metal ion, and the at least one monovalent ion may be administered by a route selected from the group consisting of oral administration, parenteral administration, and respiratory tract administration.

According to this disclosure, the retinoic acid, the at least one bivalent metal ion, and the at least one monovalent ion may be prepared into a dosage form suitable for oral, parenteral, or respiratory tract administration using technology well known to those skilled in the art. Examples of the dosage form may include, but are not limited to, sterile powder, tablets, troches, lozenges, capsules, dispersible powder, granule, solutions, suspensions, emulsions, syrup, elixirs, slurry, drops, sprays, aerosols, and the like.

Examples of the parenteral administration may include, but are not limited to, intraperitoneal injection, intrapleural injection, intramuscular injection, intravenous injection, intraarterial injection, intraarticular injection, intrasynovial injection, intrathecal injection, intracranial injection and sublingual administration.

Examples of the respiratory tract administration may include, but are not limited to, nasal/intranasal administration, intrapharyngeal administration, intratracheal administration, and intrabronchial administration. In an exemplary embodiment, the retinoic acid, the at least one bivalent metal ion, and the at least one monovalent ion are nasally administered.

According to this disclosure, the retinoic acid, the at least one bivalent metal ion, and the at least one monovalent ion may be administered with a pharmaceutically acceptable carrier that is widely employed in the art of drug-manufacturing. Examples of the pharmaceutically acceptable carrier may include, but are not limited to, solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the pharmaceutically acceptable carrier are within the expertise of those skilled in the art.

The term “liposome” as used herein refers to a particle characterized by having an aqueous interior space sequestered from an outer medium by a membrane of one or more bilayers forming a vesicle. Bilayer membranes of single- or multi-lamellar vesicles are typically formed by lipids, i.e., amphiphilic molecules of synthetic or natural origin that comprise spatially separated hydrophobic and hydrophilic domains.

Exemplary liposomes may be neutrally, positively or negatively charged liposomes.

In general, bilayer membranes of a liposome suitable for the present disclosure comprise a lipid mixture typically including dialiphatic chain lipids, such as phospholipids, diglycerides, dialiphatic glycolipids, single lipids such as sphingomyelin and glycosphingolipid, steroids such as cholesterol and derivatives thereof, and combinations thereof. Examples of phospholipids include, but are not limited to, 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1-palmitoyl-2stearoyl-sn-glycero-3-phosphocholine (PSPC), 1-palmitoyl 2-oleoyl-sn-glycero-3-phosphatidylcholine (POPC), 1,2-distearoyl-sn-glycero-3-phosphocholine (DSPC), 1,2-dioleoyl-sn-glycero-3-phosphocholine (DOPC), hydrogenated soy phosphatidylcholine (HSPC), 1,2-dimyristoyl-sn-glycero-3-phospho-(1′-rac-glycerol) (sodium salt) (DMPG), 1,2-dipalmitoyl-sn-glycero-3-phospho(1′-rac-glycerol) (sodium salt) (DPPG), 1-palmitoyl-2 stearoyl-sn-glycero-3-phospho-(1′-rac-glycerol) (sodium salt) (PSPG), 1,2-distearoyl-sn-glycero-3-phospho-(1′-rac glycerol) (sodium salt) (DSPG), 1,2-dioleoyl-sn-glycero-3 phospho-(1′-rac-glycerol) (DOPE), 1,2-dimyristoyl-sn-glycero-3-phospho-L-seine (sodium salt) (DMPS), 1,2-dipaimitoyl-sn-glycero-3-phospho-L-serine (sodium salt) (DPPS), 1,2-distearoyl-sn-glycero-3-phospho-L-serine (sodium salt) (DSPS), 1,2-dioleoyl-sn-glycero-3-phospho-L serine (DOPS), 1,2-dimyristoyl-sn-glycero-3-phosphate (sodium salt) (DMPA), 1,2-dipalmitoyl-sn-glycero-3-phosphate (sodium salt) (DPPA), 1,2-distearoyl-sn-glycero-3-phosphate (sodium salt) (DSPA), 1,2-dioleoyl-sn-glycero-3-phosphate (sodium salt) (DOPA), 1,2-dipalmitoyl-9n-glycero-3-phosphoethanolamine (DPPE), 1-palmitoyl-2-oleoyl-sn-glycero-3-phosphoethanolamine (POPE), 1,2-distearoyl-sn-glycero-3-phosphoethanolamine (DSPE), 1,2 dioleoyl-sn-glycero-3-phosphoethanolamine (DOPE), 1,2-dipalmitoyl-sn-glycero-3-phospho-(1′-myo-inositol) (ammonium salt) (DPPI), 1,2-distearoyl-sn-glycero-3-phophoinositol (ammonium salt) (DSPI), 1,2-dioleoyl-sn-glycero-3-phospho-(1-myo-inositol) (ammonium salt) (DOPI), cardiolipin, L-a-phosphatidylcholine (EPC), 1,2-dioleoyl-sn-glycero-3-ethylphosphocholine (18:1 EPC), L-a-phosphatidylethanolamine (EPE), dimethyldioctadecylammonium (DDAB), 1,2-dioleoyl-3-trimethylammonium-propane (DOTAP), 1,2-di-O-octadecenyl-3-trimethylammonium propane (DOTMA), and 3β-[N—(N′,N′-dimethylaminoethane)-carbamoyl]cholesterol hydrochloride.

The suitable lipid may be a lipid mixture of one or more of the foregoing lipids, or mixtures of one or more of the foregoing lipids with one or more other lipids not listed above, membrane stabilizers or antioxidants.

The mole percent of the lipid in the bilayer membrane may be equal or less than about 50, 45, 40, 35, 30, 25, 20, 15, 10, 5 or any value or range of values there between (e.g., about 5-50%, about 5-45%, about 5-40%, about 5-35%, about 5-30%, about 5-25%, about 5-20%, about 5-15%, or about 5-10%).

The lipid of the bilayer membrane may be a mixture of a first lipid and a second lipid. The first lipid may be selected from the group consisting essentially of phosphatidylcholine (PC), HSPC, DOPC, POPC, DSPC, DPPC, DMPC, PSPC and combinations thereof, and the second lipid is selected from the group consisting essentially of a phosphatidylethanolamine, phosphatidylglycerol, PEG-DSPE, DPPG, DOPG, DOTAP, DOTMA, DDAB and combination thereof. In other embodiments, the mole percent of the first lipid in the bilayer membrane is about 50, 45, 40, 35, 30, 25, 20, 15, 10 or any value or range of values therebetween (e.g., about 5-50%, about 5-45%, about 5-40%, about 5-35%, about 5-30%, about 5-25%, about 5-20%, about 5-15%, or about 5-10%) and the mole percent of the second lipid in the bilayer membrane is between 0.1 to about 15, 14, 13, 12, 11, 10, 9, 8, 7 or any value or range of values therebetween (e.g., about 0.1-15%, about 0.1-10%, about 0.5-15%, about 0.5-10% or about 0.5-7%). The mole 0 of the first lipid, the second lipid and cholesterol in the bilayer membrane may be about 25-50%:0.1-15%:15-55%, 5-50%:0.1%-15%:10-40% or 25-50%:0.5-10%:5-20%. The first phospholipid(s) (DSPC) and second phospholipid(s) (DOPE or DDAB) may be at a molar ratio of 4:1 to 6:1.

The bilayer membrane of the liposome further comprises less than about 55 mole percentage of steroids, preferably cholesterol. The mole % of steroid (such as cholesterol) in the bilayer membrane may be about 15-55%, about 20-55%, about 25-55%, about 15-50%, about 20-50%, about 25-50%, about 15-45%, about 20-45%, about 25-45%, about 15-40%, about 20-40% or about 25-40%. The mole % of the lipid and cholesterol in the bilayer membrane may be about 25-50%:15-55%, 25-50%:20-55% or 25-50%:15-50%. The phospholipid(s) and cholesterol may be at a molar ratio of 1:1 to 3:1. The mole % of the first lipid, the second lipid and cholesterol in the bilayer membrane may be about 25-50%:0.1-15%:15-55%, 5-50%:0.1%-15%:10-40%, or 25-50%:0.5-10%:5-20%.

The liposome encapsulating a trapping agent can be prepared by any of the techniques now known or subsequently developed. For example, the multilamellar vesicle (MLV) liposomes can be directly formed by a hydrated lipid film, spray-dried powder or lyophilized cake of selected lipid compositions with trapping agent; the SUV liposomes and LUV liposomes can be sized from MLV liposomes by sonication, homogenization, microfluidization or extrusion.

The dosage and the frequency of administration of the retinoic acid, the at least one bivalent metal ion, and the at least one monovalent metal ion may vary depending on the following factors: the severity of the viral infection or illness to be treated and the weight, age, physical condition and response of the subject to be treated. The daily dosage of the aforesaid treating agents may be administered in a single dose or in several doses.

The present disclosure also provides a method for treating a disease associated with coronavirus infection, which includes administering to a subject in need thereof the aforesaid retinoic acid and the aforesaid at least one bivalent metal ion. The coronavirus infection may be caused by the aforesaid coronaviruses. The details of the administration in the treatment method are the same as those in the inhibition method described above.

According to the present disclosure, the disease associated with coronavirus infection may be coronavirus disease 2019 (COVID-19), severe acute respiratory syndrome (SARS), Middle East respiratory syndrome, acute respiratory distress syndrome (ARDS), severe lower respiratory tract illness, or influenza-like illness. In an exemplary embodiment, the disease associated with coronavirus infection is COVID-19.

The present disclosure will be further described by way of the following examples. However, it should be understood that the following examples are intended solely for the purpose of illustration and should not be construed as limiting the present disclosure in practice.

### EXAMPLES

### Example 1. In Vitro Enzymatic Assay for Evaluating Effect of Combination of Retinoic Acid and Bivalent Metal Ion Against Severe Acute Respiratory Syndrome Coronavirus 2 (SARS-CoV-2)

In order to determine whether the combination of a retinoic acid and at least one bivalent metal ion can inhibit SARS-CoV-2, the in vitro inhibitory effect of such combination on activity of SARS-CoV-2 papain-like protease (PLPro) was first assessed.

### A. Preparation of SARS-CoV-2 PLPro

The codon-optimized gene sequence encoding wild-type SARS-CoV-2 PLpro was synthesized by Biotools (New Taipei City, Taiwan) and sub-cloned into pET-21a (Novagen) vector using the NdeI and XhoI restriction sites, while the His-tag coding region (-LEHHHHHH-) was retained at the C-terminus.

The vector with the inserted SARS-CoV-2 PLPro gene was transformed into E coli BL21 (DE3) strain (Yeastern Biotech Co., Ltd., New Taipei City, Taiwan) for overexpression of PLPro therein. Cultivation was performed in LB medium (containing 1% tryptone, 0.5% yeast extract, and 1% NaCl) supplemented with ampicillin (100 μg/mL) serving as an antibiotic marker. The resultant culture was initially incubated at 37° C. with being shaken at 200 r.p.m. At an optical density at 600 nm (OD₆₀₀) between 0.6 and 0.8, isopropyl β-D-1-thiogalactopyranoside (IPTG) was added to reach a final concentration of 0.4 mM to induce the expression of PLpro. Incubation continued at 18° C. and 200 r.p.m. for 20 hours. The cells were harvested by centrifugation (5,000×g) and disrupted by sonication in a lysis buffer containing 50 mM sodium phosphate (pH 7.4), 1.0 mM DTT, 5% glycerol and 100 mM NaCl. The cell debris was then removed by centrifugation at 20,000×g for 50 minutes. The supernatant was loaded onto a 5 mL His-Trap HP column (GE Healthcare Life Sciences), and the protein therein was eluted, using a gradient of 0˜500 mM imidazole in 50 mM sodium phosphate (pH 7.4) and 100 mM NaCl. Fractions containing His-tagged SARS-CoV-2 PLpro were pooled and concentrated, using a Centricon membrane (10K cutoff, GE Healthcare Life Sciences). His-tagged SARS-CoV-2PLpro was further purified by gel filtration chromatography, using Superdex 75 gel filtration column (GE Healthcare Life Sciences) in a 50 mM sodium phosphate buffer (pH 7.4). The SARS-CoV-2PLpro concentration was determined by measuring the ultraviolet absorbance at 280 nm, using an extinction COEFFICIENT (ε280) OF 45270 M⁻¹cm⁻¹.

### B. SARS-CoV2 PLPro Activity Assay

The enzymatic activity of SARS-CoV-2 PLpro obtained in section A of this example was measured by a colorimetry-based peptide cleavage assay, using the 6-mer peptide substrate, FRLKGG-para-nitroanilide (FG6-pNA) (purity 97% by HPLC; GL Biochem Ltd., Shanghai, China). In the cleavage assay, the 6-mer peptide substrate was cleaved at the Gly-pNA bond to release free pNA, which turned the color of the solution to yellow. The enzymatic activity was determined by continuously monitoring the absorbance at 405 nm (A₄₀₅) using a 96-well microplate spectrophotometer (Epoch™ 2, Biotek) at 30° C.

Specifically, the cleavage assay was conducted in a 96-well microplate. Each of the wells of the microplate contained a 50 mM phosphate buffer (pH 7.4), and FG6-pNA was added into the respective well such that substrate solutions having various concentrations of FG6-pNA (0.1875 mM, 0.375 mM, 0.75 mM, 1.5 mM, 3.0 mM, 6.0 mM) were prepared. The assay mixture (180 μL in each well) was preincubated for 10 minutes for accurate temperature control, and the reaction was initiated by adding 20 μL of a SARS-CoV-2 PLpro solution (1.75 μM) to the assay mixture. The SARS-CoV-2 PLpro solution was prepared by admixing the SARS-CoV-2 PLpro obtained in section A of this example with a 50 mM sodium phosphate buffer (pH 7.4). The concentration of pNA released by proteolysis was calculated by measuring A₄₀₅ using an extinction coefficient (ε405) of 9800M⁻¹cm⁻¹ (A₄₀₅=9.8 at 1 mM).

The steady state enzyme kinetic parameters were obtained by fitting the initial velocity (Vo) data based on the Michaelis-Menten Equation, using the OriginPro 8.0 software (OriginLab Corporation, USA). All measurements were performed in triplicate. The data obtained are expressed as mean±standard deviation.

### Results:

The Km and kcat values are 2.50±0.03 mM and 0.85±0.01 s⁻¹, respectively. Therefore, it is verified that SARS-CoV2 PLPro having protease activity was successfully prepared in section A of this example, and could be used to perform the following SARS-CoV2 PLPro inhibition assay.

### C. SARS-CoV2 PLPro Inhibition Assay

An enzyme inhibition assay was performed in a 96-well microplate. Each of the wells of the microplate contained a 50 mM phosphate buffer (pH 7.4). SARS-CoV2 PLPro obtained in section A of this example (0.9 μM) was added into the respective well to form an enzyme solution.

The enzyme solutions in the wells were divided into three experimental groups (i.e. Experimental Groups 1 to 3), three comparative groups (i.e. Comparative Groups 1 to 3), and a control group. To the enzyme solution of the respective group, the corresponding inhibiting agent shown in Table 1 below was added to form a test mixture (with a total volume of 180 μL). Preincubation was conducted for 30 minutes.

TABLE 1

| Group | Inhibitor agent |
|---|---|
| Experimental Group 1 | Isotretinoin (50 μM) and Zn²⁺ |
| | (0.09 μM) |
| Experimental Group 2 | Isotretinoin (50 μM) and Mg²⁺ |
| | (0.9 μM) |
| Experimental Group 3 | Isotretinoin (50 μM), Zn²⁺ |
| | (0.09 μM), and Mg²⁺ (0.9 μM) |
| Comparative Group 1 | Isotretinoin (50 μM) |
| Comparative Group 2 | Mg²⁺ (0.9 μM) |
| Comparative Group 3 | Zn²⁺ (0.09 μM) |
| Control group | None |

20 μL of FG6-pNA (1.2 mM) described in section B of this example was added into the test mixture of the respective group to initiate the enzyme reaction. The enzyme reaction was allowed to proceed at 30° C. for 300 seconds. The enzymatic activity was determined by continuously monitoring the absorbance at 405 nm (A₄₀₅ using a 96-well microplate spectrophotometer (Epoch™ 2, Biotek). The reaction rate was calculated accordingly (the reaction rate is the slope of the absorbance A₄₀₅ versus time (seconds) for a total reaction time of 300 seconds).

The inhibition percentage was calculated using the following equation:

A=[1−(B/C)]×100 (I)

where
A=inhibition percentage
B=reaction rate of respective group
C=reaction rate of control group

The data obtained are expressed as mean±standard deviation.

### Results:

The inhibition percentage of all the groups is shown in Table 2 below.

TABLE 2

| Group | Inhibition percentage |
|---|---|
| Experimental Group 1 | 33.08 ± 0.58 |
| Experimental Group 2 | 25.11 ± 2.05 |
| Experimental Group 3 | 61.93 ± 0.35 |
| Comparative Group 1 | 20.63 ± 1.27 |
| Comparative Group 2 | 13.99 ± 0.11 |
| Comparative Group 3 | 19.83 ± 0.88 |
| Control group | 0 |

As show in Table 2, the inhibition percentage in each of Experimental Groups 1 to 3 was significantly higher than those in Comparative Groups 1 to 3, indicating that the combination of isotretinoin (13-cis-retinoic acid) with at least one divalent metal ion (Zn²⁺ alone, Mg²⁺ alone, or both of these two ions in this example) can potently inhibit the enzymatic activity of SARS-CoV2 PLpro, compared with isotretinoin, Zn²⁺, or Mg²⁺ alone. This result demonstrates that isotretinoin and divalent metal ions have a synergistic inhibitory effect on papain-like protease activity, providing important insights into the biochemical properties of the coronaviral papain-like protease family and pave the way for promising therapeutic strategies against SARS-CoV2.

### Example 2. In Vivo Animal Test for Evaluating Effect of Combination of Retinoic Acid and Bivalent Metal Ion Against SARS-CoV-2

Since the combination of a retinoic acid and at least one bivalent metal ion was proven to have in vitro inhibitory effect against SARS-CoV-2, such combination was further tested for its in vivo effect on SARS-CoV-2.

### A. Establishment of SARS-CoV-2 Animal Model

Golden Syrian hamsters (aged 5-6 weeks old and having an average weight of about 100 g) were obtained from, the National Laboratory Animal Center (Taipei, Taiwan). The hamsters were housed in an animal room under specific-pathogen-free (SPF) conditions commonly applied in the art. Furthermore, water and feed were provided ad libitum for all the hamsters. All the experiments involving the hamsters were consigned to and reviewed and approved by the Institutional Animal Care and Use Committee (IACUC) of Academia Sinica (Taiwan).

The hamsters were infected with SARS-CoV-2 (obtained from Dr. Jia-Tsrong Jan, the Genomics Research Center, Academia Sinica; WuHan wild type) (in phosphate buffered saline (PBS)) through intranasal inoculation at 1×10⁴ plaque-forming units (PFU) on 12:00 PM, so as to establish a SARS-CoV-2 animal model. The establishment of the SARS-CoV-2 animal model was confirmed (data not shown).

### B. Evaluation for In Vivo Treatment Effect of Combination of Retinoic Acid and Bivalent Metal Ion Against SARS-CoV-2 in SARS-CoV-2 Animal Model

The infected hamsters obtained in section A of this examples were divided into the following five groups (n=5 per group): a control group, two experimental groups (i.e. Experimental Groups 1 and 2), and two comparative groups (i.e. Comparative Groups 1 and 2). The treatment agent respectively used for these groups are listed in Table 3 below.

TABLE 3

| Group | Treatment agent |
|---|---|
| Experimental Group 1 | Isotretinoin and higher dosage |
| | of combination of Zn²⁺, and |
| | Mg²⁺, and K⁺ |
| Experimental Group 2 | Isotretinoin and lower dosage |
| | of combination of Zn²⁺, Mg²⁺, |
| | and K⁺ |
| Comparative Group 1 | Isotretinoin |
| Comparative Group 2 | Combination of Zn²⁺, Mg²⁺, and |
| | K⁺ |
| Control group | Buffer containing 40% ethanol, |
| | 40% Span ® 80 (sorbitan |
| | monooleate) (Sigma-Aldrich), |
| | and 20% peanut oil |

The treatment agents used for Experimental Groups 1 and 2 and Comparative Groups 1 and 2 were prepared by Taipei Medical University using a liposome through a technique commonly used in the art and as described in the detail description of the present disclosure. Since the main technical feature of the present disclosure resides in the combination of a retinoic acid and at least one bivalent metal ion, the detail of the liposome is omitted herein for the sake of brevity.

Specifically, for each hamster in Experimental Groups 1 and 2, isotretinoin was administered through nasal administration at 8:00 AM on the infection day (i.e. 4 hours before the SARS-CoV-2 infection) at a dose of 0.5 mg/kg body weight and at 8:00 PM on the infection day at a dose of 0.5 mg/kg body weight, isotretinoin was administered twice daily through nasal administration at a dose of 0.5 mg/kg body weight at 8:00 AM and 8:00 PM on the two days after the infection day, and the combination of Zn²⁺ (100 μM), Mg²⁺ (200 μM), and K⁺ (200 μM) was given once daily through nasal administration at a time ranging from 6:00 PM to 6:30 PM (i.e. 1.5 hour to 2 hours before the administration of isotretinoin at night) on the infection day and the two days thereafter at a dose of 30 μL (for Experimental Group 1) or at a dose of 15 μL (for Experimental Group 2). For each hamster in Comparative Group 1, isotretinoin was administered through nasal administration at 8:00 AM on the infection day (i.e. 4 hours before the SARS-CoV-2 infection) at a dose of 0.5 mg/kg body weight and at 8:00 PM on the infection day at a dose of 0.5 mg/kg body weight, and isotretinoin was administered twice daily through nasal administration at a dose of 0.5 mg/kg body weight at 8:00 AM and 8:00 PM on the two days after the infection day. For each hamster in Comparative Group 2, the combination of Zn²⁺ (100 μM), Mg²⁺ (200 μM), and K⁺ (200 μM) was given once daily through nasal administration at a time ranging from 6:00 PM to 6:30 PM on the infection day and the two days thereafter at a dose of 30 μL. For each hamster in the control group, the buffer was administered through nasal administration at 8:00 AM on the infection day (i.e. 4 hours before the SARS-CoV-2 infection) at a dose of 100 μL and at 8:00 PM on the infection day at a dose of 100 μL, and the buffer was administered twice daily through nasal administration at a dose of 100 μL at 8:00 AM and 8:00 PM on the two days after the infection day.

After the 3-day treatment, the hamsters were sacrificed, and the lungs thereof were collected to measure the viral load generally according to the method described in Lien (2021), Sci. Rep., 11 (1):8761. doi: 10.1038/s41598-021-88283-8. The virus titer was determined in terms of the 50% tissue culture infectious dose (TCID₅₀) using the Reed-Muench method. All the experiments with SARS-CoV-2 were conducted in the biosafety level 3 (BSL-3) laboratory and were approved by Academia Sinica (Taipei, Taiwan).

The experimental data were analyzed by Tukey's test, so as to evaluate the differences between the groups. Statistical significance is indicated by p<0.05.

### Results:

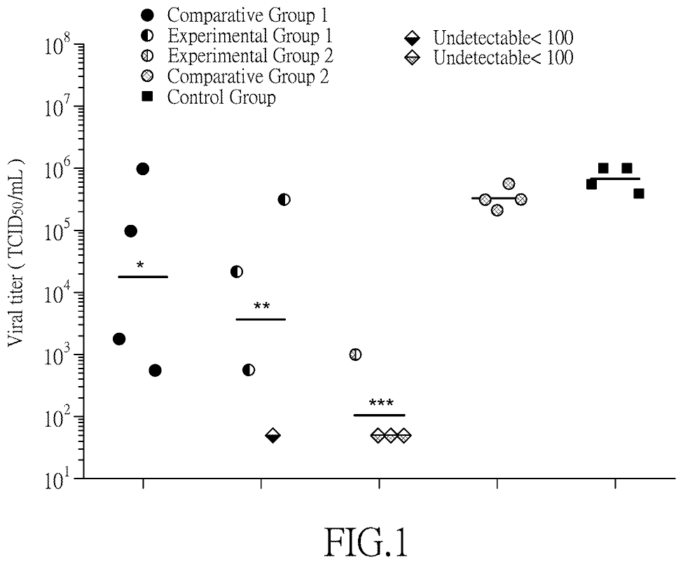

Referring to FIG. 1, TCID₅₀ of each of Experimental Groups 1 and 2 was significantly lower than those of Comparative Groups 1 and 2, revealing that the combination of isotretinoin with at least one bivalent metal ion (the combination of Zn²⁺ and Mg²⁺ in this example) and at least one monovalent metal ion (K⁺ in this example) has higher in vivo efficacy against SARS-CoV2 compared to only isotretinoin or only the combination of Zn²⁺, Mg²⁺, and K⁺.

In view of the results of Examples 1 and 2, it is verified that a retinoic acid and at least one bivalent metal ion can provide a synergistic effect on inhibiting infection and replication of SARS-CoV2 and treating a disease associated with SARS-CoV2 infection. Such combination indeed can serve as a drug-repurposing agent.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from, another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for inhibiting a coronavirus, comprising administering to a subject in need thereof a retinoic acid and at least one bivalent metal ion.

2. The method as claimed in claim 1, wherein the retinoic acid is 13-cis-retinoic acid.

3. The method as claimed in claim 1, wherein the at least one bivalent metal ion is selected from the group consisting of Zn²⁺, Mg²⁺, Cu²⁺, Mn²⁺, and combinations thereof.

4. The method as claimed in claim 3, wherein the at least one bivalent metal ion is Zn²⁺ or Mg²⁺.

5. The method as claimed in claim 3, wherein the at least one bivalent metal ion is a combination of Zn²⁺ and Mg²⁺.

6. The method as claimed in claim 1, further comprising administering to the subject at least one monovalent metal ion selected from the group consisting of K⁺, Na⁺, and a combination thereof.

7. The method as claimed in claim 1, wherein the coronavirus is selected from the group consisting of severe acute respiratory syndrome coronavirus (SARS-CoV), severe acute respiratory syndrome coronavirus 2 (SARS-CoV-2), Middle East respiratory syndrome coronavirus (MERS-CoV), human coronavirus 229E (HcoV-229E), human coronavirus OC43 (HCoV-OC43), human coronavirus NL63 (HCoV-NL63), human coronavirus HKU (HCoV-HKU1), and combinations thereof.

8. The method as claimed in claim 7, wherein the coronavirus is SARS-CoV-2.

9. The method as claimed in claim 1, wherein the retinoic acid and the at least one bivalent metal ion are administered separately, simultaneously, or sequentially.

10. The method as claimed in claim 8, wherein the at least one bivalent metal ion is administered before the retinoic acid is administered, and a time interval between administration of the at least one bivalent metal ion and that of the retinoic acid is at least one hour.

11. The method as claimed in claim 1, wherein the retinoic acid and the at least one bivalent metal ion are administered by a route selected from the group consisting of oral administration, parenteral administration, and respiratory tract administration.

12. A method for treating a disease associated with coronavirus infection, comprising administering to a subject in need thereof a retinoic acid and at least one bivalent metal ion.

13. The method as claimed in claim 12, wherein the retinoic acid is 13-cis-retinoic acid.

14. The method as claimed in claim 12, wherein the at least one bivalent metal ion is selected from the group consisting of Zn²⁺, Mg²⁺, Cu²⁺, Mn²⁺, and combinations thereof.

15. The method as claimed in claim 14, wherein the at least one bivalent metal ion is Zn²⁺ or Mg²⁺.

16. The method as claimed in claim 14, wherein the at least one bivalent metal ion is a combination of Zn²⁺ and Mg²⁺.

17. The method as claimed in claim 12, further comprising administering to the subject at least one monovalent metal ion selected from the group consisting of K⁺, Na⁺, and a combination thereof.

18. The method as claimed in claim 12, wherein the coronavirus infection is caused by a coronavirus selected from the group consisting of severe acute respiratory syndrome coronavirus (SARS-CoV), severe acute respiratory syndrome coronavirus 2 (SARS-CoV-2), Middle East respiratory syndrome coronavirus (MERS-CoV), human coronavirus 229E (HcoV-229E), human coronavirus OC43 (HCoV-OC43), human coronavirus NL63 (HCoV-NL63), human coronavirus HKU (HCoV-HKU1), and combinations thereof.

19. The method as claimed in claim 18, wherein the coronavirus is SARS-CoV-2.

20. The method as claimed in claim 12, wherein the retinoic acid and the at least one bivalent metal ion are administered separately, simultaneously, or sequentially.

21. The method as claimed in claim 18, wherein the at least one bivalent metal ion is administered before the retinoic acid is administered, and a time interval between administration of the at least one bivalent metal ion and that of the retinoic acid is at least one hour.

22. The method as claimed in claim 12, wherein the retinoic acid and the at least one bivalent metal ion are administered by a route selected from the group consisting of oral administration, parenteral administration, and respiratory tract administration.
